SEQUENCE LISTING (6 sequences)

SEQ ID NO: 1 - protein, 16 aa, Artificial Sequence
  synthetic
RDLEKHGAITSSNTAA

SEQ ID NO: 2 - protein, 6 aa, Artificial Sequence
  synthetic
FPVTPQ

SEQ ID NO: 3 - protein, 6 aa, Artificial Sequence
  synthetic
LKEKGG

SEQ ID NO: 4 - protein, 13 aa, Artificial Sequence
  synthetic
VARELHPEYFKNC

SEQ ID NO: 5 - protein, 6 aa, Artificial Sequence
  synthetic
KEKGGL

SEQ ID NO: 6 - protein, 6 aa, Artificial Sequence
  synthetic
RRSLHL

(19) United States
(12) Patent Application Publication
(10) Pub. No.: US 2019/0010217 A1
(43) Pub. Date: Jan. 10, 2019

(54) MOBILIZING HIV-INFECTED CELLS FROM LYMPHATIC RESERVOIRS

(71) Applicant: INDIANA UNIVERSITY RESEARCH & TECHNOLOGY CORPORATION, Indianapolis, IN (US)

(72) Inventors: Matthias Alexander Clauss, Indianapolis, IN (US); Bernhard Fidelis Maier, Indianapolis, IN (US)

(21) Appl. No.: 16/080,382

(22) PCT Filed: Feb. 28, 2017
(86) PCT No.: PCT/US17/19897

§ 371 Date: Aug. 28, 2018

Related U.S. Application Data
(60) Provisional application No. 62/301,207, filed on Feb. 29, 2016.

(51) Int. Cl. C07K 16/10; A61K 39/42; A61K 45/06

(52) U.S. Cl. CPC C07K 16/1072; A61K 2039/505; A61K 45/06; A61K 39/42

(57) ABSTRACT

Provided herein are methods of treating HIV infection, including retention of HIV+ T cells in viral reservoirs such as lymph nodes. More particularly, provided herein are methods in which an effective amount of a HIV Nef pathway inhibitor (e.g., anti-Nef agent) is administered to a subject in need thereof, whereby administration of the inhibitor treats HIV infection in the subject, decreases retention of HIV+ T cells in lymph nodes, and increases migration of HIV+ T cells from lymph nodes.

### CROSS-REFERENCES TO RELATED APPLICATIONS

This application claims priority to U.S. Provisional Application No. 62/301,207 filed on Feb. 29, 2016, which is incorporated by reference in its entirety.

### STATEMENT REGARDING FEDERALLY FUNDED RESEARCH

This invention was made with government support under HL129843 awarded by the National Institutes of Health. The government has certain rights in the invention.

### BACKGROUND

Antiretroviral therapy (ART) can dramatically improve the clinical outcome for HIV-infected individuals who have access to these drugs. However, one of the barriers to complete eradication of HIV from an infected individual is the persistence of HIV infection in viral reservoirs in the peripheral blood and lymphoid tissues despite the suppression of plasma viremia. Elimination of these viral reservoirs is an important treatment goal not achieved by current antiviral therapies. There is evidence that HIV infection slows the migration of T cells out of lymph nodes, creating a hide out to evade immune attack and antiretroviral therapy (ART).

There remains a need in the art for a better understanding of the etiopathology of HIV infection. In addition, there remains a need in the art for improved methods for eradicating HIV infection, for slowing or reversing HIV-associated disease progression, and for identifying candidate therapeutic agents to treat HIV+ patients.

### SUMMARY OF THE DISCLOSURE

In a first aspect, provided herein is a method of treating HIV infection, the method comprising: administering a therapeutically effective dose of an anti-Nef agent to a patient in need thereof; wherein the anti-Nef agent specifically targets Nef or Nef-mediated signaling, and wherein administering the therapeutic anti-Nef agent treats the HIV infection. The anti-Nef agent can be selected from the group consisting of an anti-Nef antibody, a small molecule inhibitor of Nef or Nef-mediated signaling, and a small nucleic acid modulator of Nef or Nef-mediated signaling. Administration of the anti-Nef agent can decrease retention of HIV+ T cells in lymph nodes in the subject. Administration of the anti-Nef agent can promote movement of HIV+ T cells into lymph and bloodstream.

The anti-Nef agent can further comprises an antibody against a cell surface protein. In some cases, the anti-Nef agent is an agent capable of in vivo expression of an anti-Nef siRNA. The agent can comprise a viral vector.

In some cases, the method further comprises administering to the patient an antiviral agent. The antiviral agent can be an anti-HIV agent selected from the group consisting of reverse transcriptase inhibitors, protease inhibitors viral maturation inhibitors, agents targeting the expression of HIV genes, agents targeting key host cell genes and gene products involved in HIV replication, iRNA agents, antisense RNA, vectors expressing iRNA agents or antisense RNA, PNA and antiviral antibodies.

In another aspect, provided herein is a pharmaceutical composition for treating HIV infection, where the pharmaceutical composition comprises: (1) an anti-Nef agent; and (2) a pharmaceutically acceptable carrier, wherein the anti-Nef agent reduces the level of Nef gene expression or a biological activity of the Nef protein in cells infected by HIV, resulting in migration of HIV+ T cells from lymph nodes.

In yet another aspect, provided herein is a method for improving CD4+ T cell mediated immunity of a HIV positive patient, where the method comprises administering to the patient a therapeutically effective amount of a therapeutic anti-Nef agent, where said therapeutic anti-Nef agent improves CD4+ T cell mediated immunity. The anti-Nef agent can be selected from the group consisting of an anti-Nef antibody, a small molecule inhibitor of Nef or Nef-mediated signaling, and a small nucleic acid modulator of Nef or Nef-mediated signaling. Administration of the anti-Nef agent can decrease retention of HIV+ T cells in lymph nodes in the subject. Administration of the anti-Nef agent can promote movement of HIV+ T cells into lymph and bloodstream. The anti-Nef agent can further comprise an antibody against a cell surface protein. The anti-Nef agent can be an agent capable of in vivo expression of an anti-Nef siRNA. The agent can comprise a viral vector.

In some cases, the method further comprises administering to the patient an antiviral agent. The antiviral agent can be an anti-HIV agent selected from the group consisting of reverse transcriptase inhibitors, protease inhibitors viral maturation inhibitors, agents targeting the expression of HIV genes, agents targeting key host cell genes and gene products involved in HIV replication, iRNA agents, antisense RNA, vectors expressing iRNA agents or antisense RNA, PNA and antiviral antibodies.

### BRIEF DESCRIPTION OF THE DRAWINGS

The present invention will be better understood and features, aspects and advantages other than those set forth above will become apparent when consideration is given to the following detailed description thereof. The detailed description makes reference to the following drawings, wherein:

FIG. 1 is a schematic illustrating that Nef-dependent down-regulation of the S1P receptor subtype 1 (S1P₁) but not chemokine receptors such as CCR7, is associated with increased retention of HIV+ T cells in the lymph nodes.

FIG. 2 demonstrates that Nef mediates prolonged vascular interactions including increased adhesion and reduced transmigration, leading to prolonged retention of HIV+ T cells in lymph nodes.

FIG. 3 demonstrates that the surface receptor S1P₁ is down-regulated by HIV-Nef-induced cell signaling. Surface S1P₁ receptor expression was determined using FACS analysis.

FIG. 4 demonstrates that S1P₁ receptor expression is down-regulated by HIV-Nef-induced cell signaling.

FIG. 5 illustrates that statin treatment reduces T cell adhesion. *=p<0.05; ***=p<0.001. Nef =Nef-ER T1 cells. Con=T1 cells.

FIG. 6A is a schematic of the modular design of TANGO constructs (top) and the general scheme for the β-arrestin (TANGO) recruitment assay (bottom).

FIG. 6B demonstrates surface expression as shown by immunofluorescence using an anti-FLAG antibody for two selected TANGO constructs.

FIG. 6C demonstrates concentration-response curves of a prototypical non-orphan GPCR, the neuromedin B receptor simulated by neuromedin B (NMB) in the TANGO assay. Data are shown as the mean ±SEM of typical experiments done in quadruplicate. Curves were fitted using Graphpad Prism 5.0.

FIG. 6D demonstrates the concentration-response curves of a prototypical non-orphan GPCR, the neuromedin B receptor simulated by neuromedin B (NMB) in a calcium-release assay. Data are shown as the mean ±SEM of typical experiments done in quadruplicate. Curves were fitted using Graphpad Prism 5.0.

FIG. 7 demonstrates that introduction of WT Nef into a cell activates β-arrestin.

FIG. 8 demonstrates there is an increase in β-arrestin activation when cells are treated with Nef in combination with S1P or FTY 720 (fingolimod).

FIG. 9 demonstrates that ΔSH3Nef is unable to downregulate S1P₁ in cells.

FIG. 10A demonstrates S1P₁ expression in HTLA cells treated with control plasmid. N terminal Flag-tag of S1P₁ was stained with Flag-M2 antibodies and Alexa-488-labeled secondary antibodies. Calreticulin-RFP was used for estimating co-localization of S1P₁ with ER and Golgi. Dapi was used as nuclear stain.

FIG. 10B demonstrates S1P₁ expression and perinuclear recruitment after treatment with ΔSH3-Nef plasmid. N terminal Flag-tag of S1P₁ was stained with Flag-M2 antibodies and Alexa-488-labeled secondary antibodies. Calreticulin-RFP was used for estimating co-localization of S1P₁ with ER and Golgi. Dapi was used as nuclear stain.

FIG. 10C demonstrates S1P₁ expression and perinuclear recruitment after transfection with Nef and pS1P₁-Tango. N terminal Flag-tag of S1P₁ was stained with Flag-M2 antibodies and Alexa-488-labeled secondary antibodies. Calreticulin-RFP was used for estimating co-localization of S1P₁ with ER and Golgi. Dapi was used as nuclear stain.

### DETAILED DESCRIPTION OF THE DISCLOSURE

All publications, patents, and patent applications mentioned in this specification are herein incorporated by reference in their entirety as if each individual publication, patent, and patent application was specifically and individually indicated to be incorporated by reference.

Most lymphocytes that migrate to the lymph nodes enter from the peripheral blood. Although various leukocyte cell types are found in the arteries of lymph nodes, only lymphocytes can interact with and extravasate through high endothelial venules (HEVs) to migrate into the lymph-node parenchyma. See, for review, Miyasaka & Tanaka, Nat. Rev. Immunology 4:360-370 (2004). T cell interactions with specific receptors/ligands on lymphatic and HEV endothelium are necessary for the egress of T cells from peripheral lymph nodes into the circulation. The compositions and methods provided herein are based at least in part on the inventors' discovery of HIV-induced molecular mechanisms that prevent the egress of HIV-infected T cells leading to the maintenance of HIV reservoirs in lymph nodes. In particular, HIV protein Nef was identified as an essential molecular player in the retention of HIV+ T cells in lymph nodes. By targeting HIV-Nef induced retention pathways, we can promote mobilization of HIV-infected T cells from their lymphatic reservoirs and force them to circulate in lymph and blood where they become subject to immune attack by cytotoxic T cells and anti-retroviral therapy. This process could eventually shift immune response dynamics to favor complete eradication of HIV.

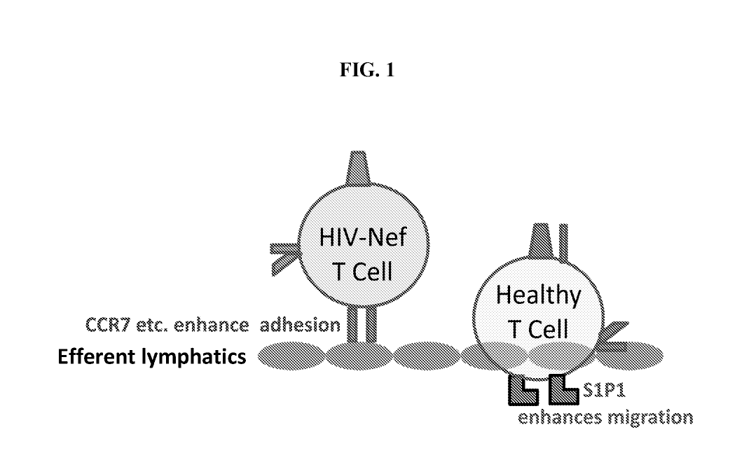

Accordingly, in a first aspect, provided herein are methods for treating HIV infection. The method includes administering to a subject in need of such treatment an effective amount of an anti-Nef agent. In some cases, the method further includes administering to the subject an effective amount of an antiviral agent. As used herein, the term “anti-Nef agent” refers to any agent that is capable of reducing the level of Nef gene expression or a biological activity of the Nef protein. The term “gene expression” as used herein refers to the process of transcription of mRNA from a coding sequence, translation of mRNA into a polypeptide, and post-translational modifications such as phosphorylation and glycosylation. A person of ordinary skill in the art would understand that an anti-Nef agent may also have anti-S1P₁ activity. Examples of anti-Nef agents include without limitation iRNA agents, antisense RNA, vectors expressing iRNA agents, or antisense RNA, PNA, anti-Nef antibodies, small molecules that target Nef and S1P₁ interactions (see FIG. 1). These agents also include the agents attached to, complexed with, inserted into, or otherwise associated with the agents that target the anti-Nef agents to particular cell types or alter the metabolic properties, pharmacokinetic characteristics, or other characteristics of the anti-Nef agents.

Preferably, compounds suitable for use as anti-Nef agents according to the methods described herein include (without limitation) those which (i) inhibit Nef activity by targeting SH3 binding domains (proline rich sequences as described by the literature as SH3 binding motifs); (ii) increase cell surface expression of the S1P receptor subtype 1 (S1P₁); (iii) modulate cytosolic S1P₁ phosphorylation; (iv) inhibit binding of Nef to S1P₁ receptor; (v) decrease T cell adhesion to lymphatic and HEV endothelium; or a combination thereof. Suitable compounds include, without limitation, antibodies, peptides, small nucleic acid modulator, and small molecular weight compounds as described in the following paragraphs.

As used herein, the term “effective amount” refers to an amount effective, at dosages and for periods of time necessary, to achieve the desired result, e.g., sufficient to inhibit gene expression or protein activity of Nef to a desired level. The effective amount of anti-Nef agent may vary according to factors such as the disease state, age, and weight of the subject, and the ability of the particular agent or agents to elicit a desired response in the subject. Dosage regimens may be adjusted to provide the optimum therapeutic response. An effective amount is also one in which any toxic or detrimental effects (e.g., side effects) of the agent(s) are outweighed by the therapeutically beneficial effects. Toxicity and therapeutic efficacy of such agents can be determined by standard pharmaceutical procedures in cell cultures or experimental animals, e.g., for determining the LD50 (the dose lethal to 50% of the population) and the ED5% (the dose therapeutically effective in 50% of the population). The dose ratio between toxic and therapeutic effects is the therapeutic index and it can be expressed as the ratio LD₅₀/ED₅₀. Agents which exhibit large therapeutic indices are preferred. While agents that exhibit toxic side effects may be used, care should be taken to design a delivery system that targets such compounds to the site of affected tissue in order to minimize potential damage to uninfected cells and, thereby, reduce side effects.

Antibodies

In some cases, the anti-Nef agent is an antibody that targets and neutralizes circulating Nef and/or molecules downstream of Nef that are associated with Nef-induced T cell retention. The term “antibody”, as used herein, is defined as an immunoglobulin that has specific binding sites to combine with an antigen. As used herein, the terms “antibody” and “antibodies” are synonymous with “immunoglobulin” and “immunoglobulins,” and the terms are used interchangeably herein. The terms “antibody” and “antibodies” include whole immunoglobulins including, without limitation, polyclonal antibodies or monoclonal antibodies (mAbs). The term “antibody” is used in the broadest possible sense and may include without limitation an antibody, a recombinant antibody, a genetically engineered antibody, a chimeric antibody, a monospecific antibody, a bispecific antibody, a multispecific antibody, a chimeric antibody, a heteroantibody, a monoclonal antibody, a polyclonal antibody, a camelized antibody, a deimmunized antibody, a humanized antibody and an anti-idiotypic antibody. The term “antibody” may also include but is not limited to an antibody fragment such as at least a portion of an intact antibody, for instance, the antigen binding variable region. Examples of antibody fragments include Fv, Fab, Fab′, F(ab′), F(ab′)₂, Fv fragment, diabody, linear antibody, single-chain antibody molecule, multispecific antibody, and/or other antigen binding sequences of an antibody.

In exemplary embodiments, neutralizing anti-Nef antibodies are antibodies (or derivatives thereof) specific for the following epitopes: 3D12 (RDLEKHGAITSSNTAA; SEQ ID NO:1); SN20 (FPVTPQ; SEQ ID NO:2); SN41 (LKEKGG; SEQ ID NO3); EHI (VARELHPEYFKNC; SEQ ID NO:4), and Src-Ban_2 (KEKGGL; SEQ ID NO:5).

In one example, the neutralizing antibodies are monoclonal antibodies that target the SH3 biding site of HIV-Nef protein, specifically the monoclonal antibodies are specific to the epitope FPVTPQ (SEQ ID NO:2) or KEKGGL (SEQ ID NO:5). In some embodiments, the methods may use a combination of at least two monoclonal antibodies. In one embodiment, the two monoclonal antibodies comprise a monoclonal antibody specific to epitope FPVTPQ (SEQ ID NO:2) and a second monoclonal antibody specific to epitope KEKGGL (SEQ ID NO:5)

In some cases, an anti-Nef agent is a single-domain antibody-SH3 fusion. For example, Bouchet et al. describe a single-domain antibody (sdAb) targeting Nef and inhibiting many, but not all, of its biological activities in CD4-positive T lymphocytes (J. Virology (2012) p. 4856-67). In some cases, an anti-Nef agent is an antibody fused to a peptide sequence for cell membrane permeability.

The term antibody encompasses various broad classes of polypeptides that can be distinguished biochemically. Those skilled in the art will appreciate that heavy chains are classified as gamma, mu, alpha, delta, or epsilon (γ, μ, α, δ, ϵ v) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization.

Antibodies appropriate for the present invention also include antibody fragments or modified products thereof, provided that they can be suitably used in the present invention. Appropriate antibody fragments comprise at least one variable domain of an immunoglobulin, such as single variable domains Fv (Skerra & Pluckthun, Science 240:1038-41 (1988)), scFv (Bird et al., Science 242:423-26 (1988); Huston et al., Proc. Natl. Acad. Sci. USA 85:5879-83 (1988)), Fab, (Fab′)₂ or other proteolytic fragments. The terms “antibody” and “antibodies” further include chimeric antibodies; human and humanized antibodies; recombinant and engineered antibodies, conjugated antibodies, and fragments thereof. Humanized antibodies are antibodies wherein the complementarity determining regions (CDRs) of an antibody from a mammal other than human (e.g., a mouse antibody) are transferred into the CDRs of human antibodies. Chimeric and humanized antibodies can be made according to standard protocols such as those disclosed in U.S. Pat. No. 5,565,332. Other antibody formats are described in, for example, “Antibody Engineering,” McCafferty et al. (Eds.) (IRL Press 1996). Also encompassed in the invention are Nef-targeting immunoglobulins that have been conjugated or bound in some manner to various molecules including, without limitation, polyethylene glycol (PEG), radioactive substances, and drugs. Such conjugated antibodies can be obtained by chemically modifying a Nef-targeting immunoglobulin. Methods for obtaining conjugated antibodies are known and available in the art.

The antibodies of the present invention may be polyclonal or monoclonal antibodies. Preferably, the Nef-targeting immunoglobulins are monoclonal. Methods of producing polyclonal and monoclonal antibodies are known in the art and described generally, e.g., in U.S. Pat. No. 6,824,780. Typically, the antibodies of the present invention are produced recombinantly, using vectors and methods available in the art, as described further below. Human antibodies may also be generated by in vitro activated B cells (see U.S. Pat. Nos. 5,567,610 and 5,229,275). Human antibodies may also be produced in transgenic animals (e.g., mice) that are capable of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice results in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669; U.S. Pat. No. 5,545,807; and WO 97/17852. Such animals may be genetically engineered to produce human antibodies comprising a polypeptide of the present invention.

The source of the antibodies described herein is not particularly restricted in the present invention; however, the antibodies are preferably derived from mammals, and more preferably derived from humans. Monoclonal antibodies appropriate for the present invention can be prepared by standard hybridoma methods. For example, standard hybridoma methods employ differential binding assays to ensure that the resulting monoclonal antibodies are specific for a Nef polypeptide and do not show cross-reactivity between related proteins. Alternatively, monoclonal antibodies appropriate for the present invention can be prepared using antibody engineering methods such as phage display. Methods for obtaining highly specific antibodies from antibody phage display libraries are known in the art, and several phage antibody libraries are commercially available from, for example, MorphoSys (Martinsried, Germany), Cambridge Antibody Technology (Cambridge UK) and Dyax (Cambridge Mass.). Suitable phage display methods are described, for example, in U.S. Pat. Nos. 6,300,064 and 5,969,108, and in “Antibody Engineering,” McCafferty et al. (Eds.) (IRL Press 1996)). Once the antibody heavy and light chain genes are recovered from the phage antibodies, antibodies in any suitable format may be prepared for use according to the present invention, e.g., whole antibodies, Fab fragments, scFv, etc.

Antibodies disclosed herein may be from any animal origin including birds and mammals. Preferably, the antibodies are human, murine, donkey, rabbit, goat, guinea pig, camel, llama, horse, or chicken antibodies.

Polyclonal antibodies appropriate for the present invention can be prepared by may also be prepared using traditional animal-based methods. For example, an appropriate animal can be immunized using a polypeptide immunogen (e.g., peptide of Nef). Polypeptide antibody titers in the immunized animal can be monitored over time by standard techniques, such as with an enzyme linked immunosorbent assay (ELISA) using immobilized polypeptide. Antibodies specific to the antigen can be isolated from the mammal (e.g., from the blood) and further purified by techniques known to those practicing in the art including, for example, protein A chromatography to obtain the IgG fraction. In some cases, at an appropriate time after immunization (e.g., when the antibody titers are highest) antibody-producing cells can be obtained from the animal and used to prepare monoclonal antibodies.

As used herein, the terms “epitope” or “antigenic determinant” refer to a site on an antigen (e.g., on Nef) to which an immunoglobulin or antibody specifically binds. Generally, an epitope includes at least 3, 4, 5, 6, 7, 8, 9, 10, 11, 12, 13, 14, or 15 consecutive or non-consecutive amino acids in a unique spatial conformation. See, e.g., Epitope Mapping Protocols in Methods in Molecular Biology, Vol. 66, G. E. Morris, Ed. (1996). As used herein, the terms “specific binding,” “selective binding,” “selectively binds,” and “specifically binds,” refer to an antibody binding to an epitope via its antigen-binding domain, and that the binding entails some complementarity between the antigen-binding domain and the epitope. Generally, an antibody specifically or selectively binds with an affinity (generally represented by the dissociation constant K_{D}) of approximately less than 10⁻⁷ M, such as approximately less than 10⁻⁸ M, 10⁻⁹ M, or 10⁻¹⁰ M, or lower. As used herein, the term “affinity” denotes the strength of the sum total of non-covalent interactions between a single binding site of a molecule (e.g., a peptide, polypeptide, or antibody) and its binding partner (e.g., a target or an antigen). Unless indicated otherwise, as used herein, “binding affinity” refers to intrinsic binding affinity which reflects a 1:1 interaction between members of a binding pair (e.g., between a peptide and its target, or between an antibody and its antigen). The terms “K_{D}” and “K_{d}” are synonymous and refer to the dissociation equilibrium constant of a particular molecule X-binding partner Y interaction. Affinities of antibodies can be readily determined using methods known in the art such as surface plasmon resonance. Other conventional techniques for determining antibody affinities are known in the art, such as those described by Scatchard et al. (Ann. N.Y. Acad. Sci. USA 51:660 (1949)). Binding properties of an antibody to antigens, cells or tissues thereof may generally be determined and assessed using immunodetection methods including, for example, immunofluorescence-based assays, such as immuno-histochemistry (IHC), and/or fluorescence-activated cell sorting (FACS).

In exemplary embodiments, antibodies of the present invention bind to HIV Nef with a dissociation equilibrium constant (K_{D}) of less than approximately 10⁻⁷ M, such as approximately less than 10⁻⁸ M, 10⁻⁹ M, or 10⁻¹⁰ M, or lower.

Monoclonal antibodies can be obtained by hybridoma technology, which is the process of producing hybrid cell lines by fusing an antibody-producing B cell with a myeloma cell that can grow in tissue culture. The resulting hybridoma line produces a monoclonal antibody of a single specificity.

In some cases, antibodies useful for the methods provided herein include antibody derivatives such as peptide antibodies fused to peptide sequences that render the antibodies cell membrane permeable.

Small Molecules

In some cases, an anti-Nef agent is a small molecular weight agent capable of interfering with Nef-induced T cell adhesion or retention and/or capable of modulating phosphorylation of S1P₁ receptor. For example, Fingolimod (FTY720/Gilenya; Novartis) is an orally active immunomodulatory drug approved by the FDA for the treatment of multiple sclerosis. In animal models and in humans, fingolimod reduces peripheral blood lymphocyte counts, affecting CD4+ T cells, CD8+ T cells, and B cells, and it was speculated that the drug might accelerate the homing of lymphocytes into lymph nodes. See Brinkmann et al., Nature Rev. Drug Discovery 89:883-897 (2010). It was observed that pretreatment of lymphocytes with Pertussis toxin (PTX) to block G protein-coupled receptors (GPCRs)-Gαi signaling prevented the retention of T cells in lymph nodes by fingolimod. See Brinkmann et al., Trends Pharmacol. Sci. 21:49-52 (2000); Brinkmann et al., Transplant. Proc. 33:3078-3080 (2001). Fingolimod-related compounds that are expected to be useful anti-Nef/Nef-mediated signaling agents include, without limitation, VPC44116, KRP-203, AUY954, CYM-5442, SEW2871, W146, VPC44116, and VPC23019. See, e.g., Marsolais & Rosen, Nat. Rev. Drug Discovery 8(4):297-307 (2009).

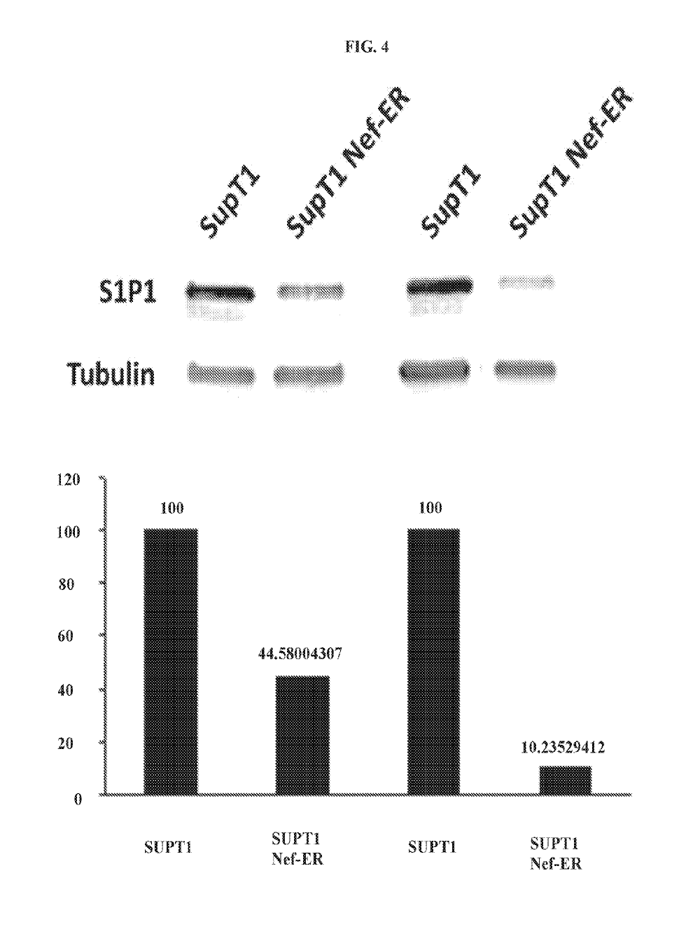

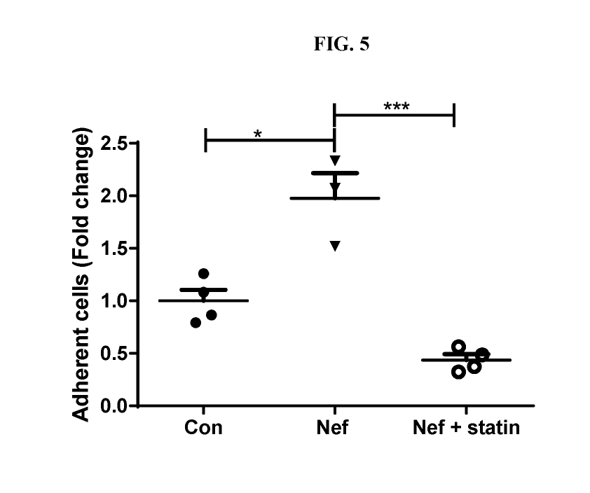

In some cases, small molecule inhibitors can specifically target S1P₁ (alternatively known as EDG1). S1P₁ has been shown to operate in conjunction with EDG3 to control signaling pathways necessary for endothelial cell migration (Lee et al. (1999) Cell 99:301-312). In particular, S1P activity is mediated by its binding to and activation of G-protein-coupled S1P receptors expressed at the endothelial cell surface. Receptors S1P₁ and S1P₃ represent major receptors for S1P expressed in endothelial cells. Statins have been shown to induce S1P₁ receptors and potentiate responses of endothelial cells to high-density lipoprotein (HDL)-associated sphingolipids (Igarashi et al., British Journal of Pharmacology (2007) 150, 470-479). Accordingly, in some cases, the methods provided herein further comprise administering one or more statins to upregulate S1P₁ receptor expression in lymphatic and HEV endothelium. Statins appropriate for use according to the methods provided herein include, without limitation, pitavastatin, pravastatin, atorvastatin, fluvastatin, and rosuvastatin while lovastatin, and simvastatin are contraindicated for combination with protease inhibitors as part of combined antiretroviral therapy (cART) for HIV patients (Coffey, UCSF HIV InSite (2012); available at hivinsite.ucsf.edu/InSite?page=md-rr-30 on the World Wide Web). As shown in FIG. 5, statin treatment reduces adhesion of HIV-Nef expressing T cells.

Changes in S1P₁ receptor expression can be assessed using any appropriate method. For example, surface S1P receptor expression can be assessed using FACS analysis.

iRNA Agents

In some cases, an anti-Nef agent is a small nucleic acid modulator of Nef gene or protein expression, or a vector expressing a small nucleic acid modulator of Nef induced retention pathways, e.g. DNA or miRNA vectors. As used herein, the term “iRNA agent” refers to small nucleic acid molecules used for RNA interference (RNAi), such as short interfering RNA (siRNA), double-stranded RNA (dsRNA), microRNA (miRNA) and short hairpin RNA (shRNA) molecules. The iRNA agents can be unmodified or chemically-modified nucleic acid molecules. The iRNA agents can be chemically synthesized or expressed from a vector or enzymatically synthesized. The use of a chemically-modified iRNA agent can improve one or more properties of an iRNA agent through increased resistance to degradation, increased specificity to target moieties, improved cellular uptake, and the like. The term “antisense RNA,” as used herein, refers to a nucleotide sequence that comprises a sequence substantially complementary to the whole or a part of an mRNA molecule and is capable of binding to the mRNA.

Vectors expressing iRNA agents or antisense RNA include, but are not limited to non-viral vectors and viral vectors. Non-viral vectors typically include a plasmid having a circular double stranded DNA into which additional DNA segments can be introduced. The non-viral vector may be in the form of naked DNA, polycationic condensed DNA linked or unlinked to inactivated virus, ligand linked DNA, and liposome-DNA conjugates. Viral vectors include, but are not limited to, retrovirus, adenovirus, adeno-associated virus (AAV), herpesvirus, and alphavirus vectors. The viral vectors can also be astrovirus, coronavirus, orthomyxovirus, papovavirus, paramyxovirus, parvovirus, picornavirus, poxvirus, or togavirus vectors.

In one embodiment, short interfering RNAs (siRNA) are used as an anti-Nef agent. siRNAs are dsRNAs having 19-25 nucleotides. siRNAs can be produced endogenously by degradation of longer dsRNA molecules by an RNase III-related nuclease called Dicer. siRNAs can also be introduced into a cell exogenously or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNA are duplexes of short mixed oligonucleotides which can include, for example, 19 nucleotides with symmetric 2 dinucleotide 3′ overhangs. Using synthetic 21 bp siRNA duplexes (19 RNA bases followed by a UU or dTdT 3′ overhang), sequence specific gene silencing can be achieved in mammalian cells. These siRNAs can specifically suppress targeted gene translation in mammalian cells without activation of DNA-dependent protein kinase (PKR) by longer dsRNA, which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression, since they deploy a short AT rich transcription termination site that leads to the addition of 2 bp overhangs (UU) to hairpin siRNAs—a feature that is helpful for siRNA function. Recent approaches used to selectively deliver RNAi to particular cell types include liposomal nanoparticles containing siRNAs incorporating antibodies against cell surface proteins, such as integrins (Peer D, et al. Proc Natl Acad Sci USA 2007, 104:4095-100), AAV vector systems, lentiviral vector systems (including HIV-based lentiviral vector systems) (Tiscornia G, et al. Proc Natl Acad Sci USA 2003, 100:1844-1858, Banerjea A, et al. Mol Ther 2003, 8:62-71, Li M and Rossi J J. Methods Mol Biol 2008, 433:287-299). SiRNAs complexed with single chain antibodies (scFvs) against cell surface proteins modified to have a polylysine tail that binds the RNAs can be targeted specifically to lymphocytic cells bearing the surface antigen recognized by the scFvs (Kumar P, et al. Cell 2008, 134:577-86).

In one aspect, provided herein are methods of treating a disease, condition, or disorder in a subject by inhibiting Nef and Nef-mediated signaling. For example, the present invention provides methods comprising administering to a subject in need thereof an inhibitor of Nef, whereby the disease, condition, or disorder is treated. As used herein, the terms “treating,” “treat,” and “treatment” refer to the management and care of a patient for the purpose of combating the disease, condition, or disorder. The terms embrace both preventative, i.e., prophylactic, and palliative treatments. In some cases, the term “treated” refers to any beneficial effect on progression of a disease or condition. Beneficial effects can include reversing, alleviating, inhibiting the progress of, preventing, or reducing the likelihood of the disease or condition to which the term applies or one or more symptoms or manifestations of such a disease or condition. Where the disease or condition is HIV infection or a HIV-associated condition, treating can refer to the management and care of a patient for the purpose of combating HIV, and can include reversing, alleviating, inhibiting the progress of, preventing or reducing the likelihood of, or lessening the severity of any aspect of the HIV infection or HIV-associated condition. A therapeutic beneficial effect on the health and well-being of a patient includes, but it not limited to: (1) eradicating, fully or in part, the HIV infection; (2) slowing the progress of the HIV infection; (3) increasing responsiveness of the patient to ART; or (4) increasing T cell counts in the patient. As used herein, the terms “preventing” and “prevent” refer not only to a complete prevention of a certain disease or condition, but also to partially or substantially attenuating, reducing the risk of, or delaying the development or recurrence of the disease or condition to which the term applies.

As used herein, the term “subject” refers to an individual having, suspected of having, or susceptible to having a disease or condition such as HIV infection. By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

The anti-Nef agent may be administered via commonly used administrative routes such as parenteral administration (e.g., intravenous, intramuscular, intraperitoneal, intradermal, and subcutaneous administration), enteral administration (e.g., oral and rectal administration), and topical administration (e.g., transdermal, inhalational, intranasal and vaginal administration). In one embodiment, the anti-Nef agent is administered prior to the administration of an antiviral agent. In another embodiment, the anti-Nef agent is administered concurrently with the administration of an antiviral agent. As used herein, the term “antiviral agent” refers to an agent (compound or biological) that is effective to inhibit the formation and/or replication of HIV in a mammal. Examples of antiviral agents include, but are not limited to, reverse transcriptase inhibitors such as azidothymidine (AZT), 2′,3′-dideoxyinosine (DDI), 2′,3′-didexoycytidine (DDC), didehydrothymidine (d4T), 2′-deoxy-3′-thiacytidine (3TC), abacavir succinate, and tenofovir disoproxil fumarate, nevirapine, delavirdine and efavirenz; protease inhibitors such as saquinavir, saquinavir mesylate, ritonavir, lopinavir, indinavir, nelfinavir mesylate, amprenavir, fosamprenavir, tipranavir, atazanavir, entry inhibitors such as maraviroc, vicriviroc, enfuvirtide, viral maturation inhibitors, agents targeting the expression of HIV genes, agents targeting key host cell genes and gene products involved in HIV replication, and other anti-HIV agents, iRNA agents, antisense RNA, vectors expressing iRNA agents or antisense RNA, PNA and antiviral antibodies.

Another aspect of the present invention relates to a pharmaceutical composition for treating HIV infection. The pharmaceutical composition contains (1) an anti-Nef agent as described herein, and (2) a pharmaceutically acceptable carrier. A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism. As used herein, a “pharmaceutically acceptable carrier” refers to a carrier or diluent that does not cause significant irritation to an organism and does not abrogate the biological activity and properties of the administered compound.

Any appropriate route or mode of administration to the subject can be employed according to a method provided herein. In exemplary embodiments, an inhibitor of Nef or Nef-mediated signaling is administered to a subject as a pharmaceutical composition and in an effective amount to treat and/or prevent a disorder as described herein. Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example the disclosed compounds or combinations thereof, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

In treating HIV infection, useful anti-Nef agent concentrations in the composition can range from about 5 mg/ml to about 0.00005 mg/ml. Effective blood plasma levels are expected to range from about 10⁻⁹ M to about 10⁻¹⁷M. The inventors specifically contemplate the use of all concentrations within these ranges depending on the surrounding circumstances. Different anti-Nef agent concentration levels will be optimal as one balances drug potency and adverse side effects.

In another aspect, provided herein are methods for ameliorating or abolishing HIV-Nef-induced retention of HIV+ T cells in lymph nodes that constitutes a persisting viral reservoir. Generally, the methods comprise neutralizing HIV Nef or Nef-mediated signaling, thereby decreasing retention of HIV+ T cells in lymph nodes and promoting movement of HIV+ T cells into circulating lymph and blood. Without being bound to any particular theory or mechanism, it is expected that movement of HIV+ T cells from lymph node “viral reservoirs” renders the HIV+ cells more susceptible to attack from the immune system and more available for anti-retroviral therapies.

In another aspect, provided herein are methods of screening to identify specific inhibitors of the Nef-induced pathway of T cell retention—inhibitors that are useful to promote movement of HIV-infected T cells out of their lymphatic reservoirs and, consequently, eradication of HIV by the immune system. Accordingly, the present invention provides methods for identifying candidate therapeutic agents to treat an HIV infection or HIV-associated disease, to slow or halt HIV progression, to alter a HIV disease mechanism, or to correct an observed HIV infection phenotype. For example, methods of the present invention can comprise testing compounds for their ability to reduce HIV+ T cell adhesion to lymphatic and HEV endothelium, to restore migration of HIV+ T cells from lymph nodes, or to sensitize a patient having an HIV infection to treatment using ART.

In some cases, the present invention provides a method of evaluating a candidate Nef inhibitor, where the method comprises the steps of contacting a candidate Nef inhibitor to HIV+ T cells co-cultured with endothelial cells, and evaluating the contacted HIV+ cells for an effect of the agent on adhesion to the endothelial cells. In some embodiments, the method will include evaluating the effect of the candidate Nef inhibitor relative to HIV+ cells that have not contacted the candidate Nef inhibitor.

In another instance, the present invention provides a method of evaluating S1P₁ agonists that that can upregulation S1P₁ in HIV infected cells. In some embodiments, this agonist can be used in the methods of the present invention. As described in Luttrell et al., Journal of Cell Science 115, 445-465 (2002) (incorporated by reference in entirety) beta-arrestin plays a role in S1P₁ regulation. Beta-arrestins have been shown to play a role in the desensitization, sequestration and intracellular trafficking of GPCRs from the surface to endosomal or endocytic vesicles for degradation or recycled to the plasma membrane. See Luttrell et al. Beta arrestins comprise two major functional domains, an N-terminal domain responsible for recognition of activated GPCRs and a C terminal domain responsible for secondary receptor recognition. Id.). The functionally identified A and B domains correspond approximately to the N and C domains identified crystallographically. The R2 domain contains the primary site of β-arrestin 1 phosphorylation, S412, as well as the LIEF binding motif for clathrin and the RXR binding motif for β2-adaptin (AP2). The recognition domain for inositol phospholipids(IP6) resides within the B domain. One or more PXXP motifs located within the A domain of β-arrestin 1 mediates binding to the c-Src-SH3 domain. The MAP kinase, JNK3, and possibly other MAP kinases (MAPKs), interact with β-arrestin 2 via a consensus MAP kinase recognition sequence, RRSLHL (SEQ ID NO:6), located within the B domain. Less precisely defined interactions, such as those between β-arrestin 1 (1-185) and Ask1 and Src-SH1 domains, β-arrestin 1 and NSF, and β-arrestin 2 and Mdm2, as described in Luttrell et al.

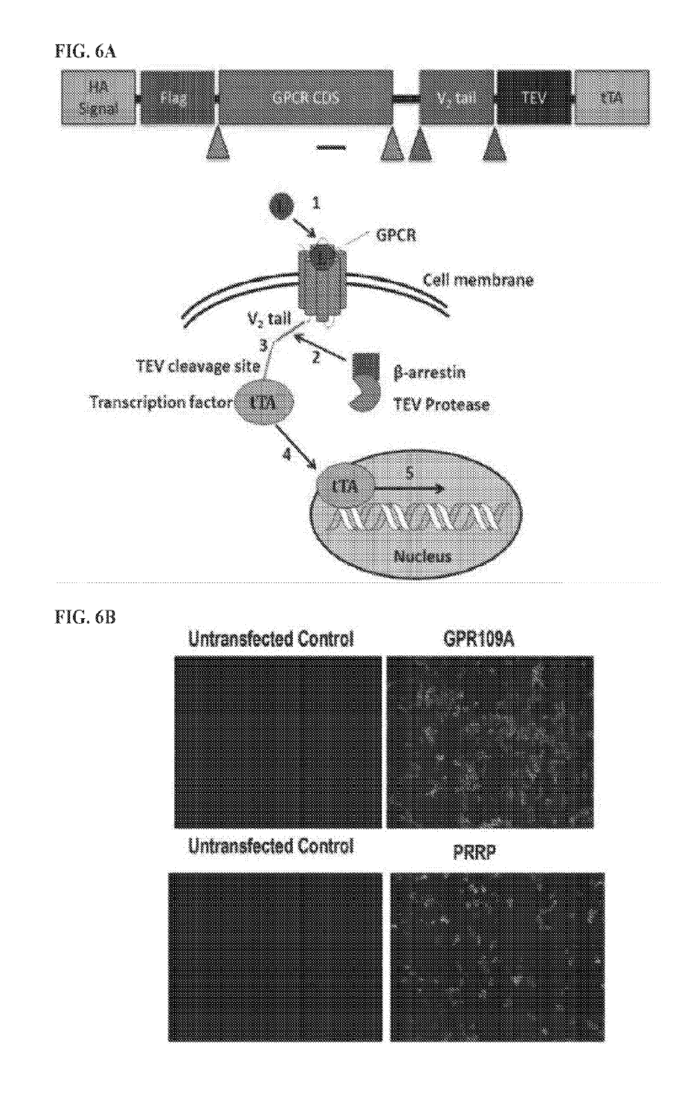

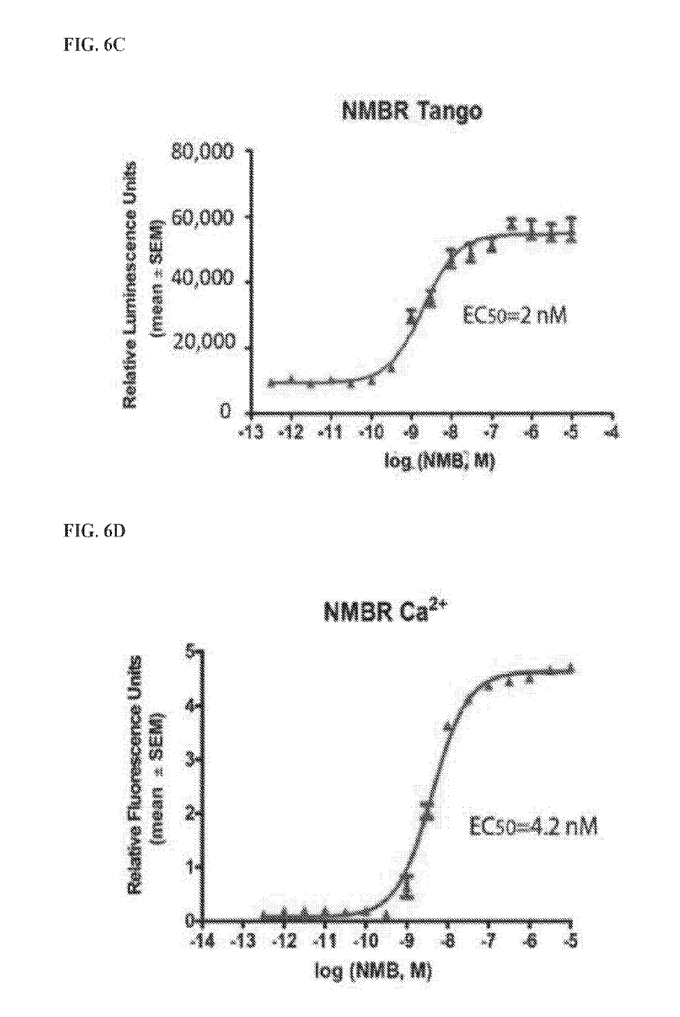

The role of beta arrestin in GPCR regulation, including S1P₁ allows for the design and use of an S1P₁ Arrestin-Tango expressing cell line (TANGO construct commercially available from AddGene) to test S1P₁ signaling and its inhibitors. FIG. 6 demonstrates the design, principle and validation of the arrestin-Tango assay. FIG. 6A shows the modular design of Tango constructs (top) and the general scheme for the β-arrestin (TANGO) recruitment assay. Upon activation of the S1P₁ by an agonist, β-arrestin is recruited to the C-terminus of the receptor. This is followed by cleavage of the GPCR fusion protein at the TEV protease site. Cleavage results in the release of the tTA transcription factor, which after transport to the nucleus, activates transcription of the luciferase reporter gene. Thus, potential SNP1 inhibitors can be screened using the S1P₁ Arresting Tango assay looking for an increase of luciferase activity indicating an increase in S1P₁ signalling.

In the specification and in the claims, the terms “including” and “comprising” are open-ended terms and should be interpreted to mean “including, but not limited to . . . . ” These terms encompass the more restrictive terms “consisting essentially of” and “consisting of. ”

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents and alternatives falling within the spirit and scope of the invention as defined by the appended claims.

The invention will be more fully understood upon consideration of the following non-limiting Examples.

### SEQUENCE LISTING STATEMENT

The application includes the sequence listing that is concurrently filed in computer readable form. This sequence listing is incorporated by reference herein.

### EXAMPLES

### Example 1

### Adhesion to Lymphatic Endothelium is Increased in the Presence of HIV-Nef

Nef was activated in Nef-ER cells by culturing them with RPMI1640+10% FBS (fetal bovine serum) and 1 μM 4-hydrotamoxifen for at least 6 hours or overnight at 37° C. For inhibitor use, the Nef-ER cells were pre-incubated for at least 12 hours with 10 μM atorvastatin. Other inhibitors may require shorter pre-incubations.

Approximately 100K HUVECS were seeded per well in a collagen-coated 6-well plate. Cells were incubated overnight in EGM2(LONZA) medium. Nef-ER cells were stained using 5 μM Calcein AM (Ref C3100MP, Fischer Scientific) in 1 mL of staining solution and incubated for 30 minutes at 37° C. The cells were centrifuged at 300 g for 5 min, and the cell pellet was resuspended in 2 mL RPMI+10% FBS. Cells were centrifuged at 300 g for 5 min and the supernatant was removed.

After staining, 1 μL of 1 mM 4-Hydrotamoxifen per ml of EGM2(LONZA) medium was added for final concentration of 1 μM. Stained T-cells were resuspended in EGM2 media at final concentration of 250,000 cells per ml. 250,000 stained T-cells were added to each well of 6-well plate of HUVECs for co-culture (with or without 10 μM atorvastatin to maintain inhibition) and incubated for 60 minutes. The plate was gently shaken one time after 30 minutes. Cells were washed very gently 3 times with PBS (phosphate buffered saline). Cells were then trypsinized and analyzed by fluorescence activated cell sorting (FACS).

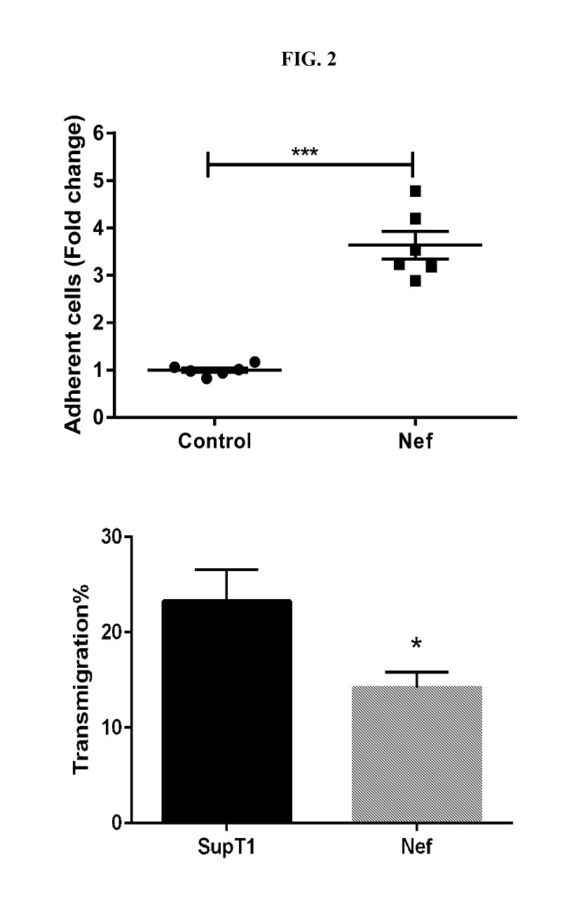

As shown in FIG. 2, Nef mediates prolonged vascular interactions including increased adhesion and reduced transmigration, leading to prolonged retention of HIV+ T cells in lymph nodes.

### Example 2

### Transmigration Assay

Approximately 20,000 endothelial cells (HUVECs lung microsvascular/lymphatic endothelial cells) were seeded 2 days prior to each experiment in a collagen-coated 24-well transwell plate. Culture medium was changed after overnight incubation, and the cells were allowed to form tight junctions for another 24 hours before each transmigration assay.

5×10⁵ SupT1 Control cells or SupT1 Nef-ER cells were labeled with 2.5 μM Calcein AM in serum-free RPMI for 30 minutes. The labeled cells were then added to endothelial cells as described above for the adhesion assay.

For the transmigration assay, EGM2 growth media was aspirated from HUVEC monolayer. HUVEC monolayer cells were washed with PBS, and then 200 μL of SupT1 control/SupT1 Nef-ER cells were added (approximately 100,000 cells/well). 300 μL of phenol red-free DMEM+10% FBS were placed in the bottom well. HUVEC monolayer+SupT1 cells-containing transwell was placed in a well containing DMEM+10% FBS and incubated for 12-18 hours.

Media in top and bottom wells separated by transwell were collected. 100 μL was added to a 96 well black clear bottom plate. Fluorescence was measured using fluorescent reader (for example, Flexstation, top read, green channel), and subtracted from blank well containing phenol red-free DMEM. Signals were calculated as follows: % transmigration=(Fluorescence in bottom well)/(Fluorescence in top well+Fluorescence in bottom well)

### Example 3

### Nef-Mediated Downregulation of S1P₁

SUP-T1 cells (a T-cell lymphoblastic lymphoma cell line) stably expressing tamoxifen-inducible Nef-ER and control SupT1 were treated for 16 hours with 1μM tamoxifen. Sample protein lysates (20 μg/lane) were separated by 4-20% gradient Tris/Glycin SDS-PAGE and blotted onto FL-PVDF membranes. S1P₁ was detected with anti-human CD363 (S1P1R) e-fluor 660 (E-bioscience). Blot signals were detected and quantified on an Licor Odyssey scanner. Samples from two experiments were analyzed.

FACS analysis of S1P₁: Nef-ER or control T1 cells were activated with 1 μM 4-hydrotamoxifen in RPMI1640+10% FBS for 16 hours, stained with anti-human S1P1R coupled E-fluor 680 from E-BioScience for one hour and then analyzed in a FACS Calibur. 30,000 cells were counted per sample.

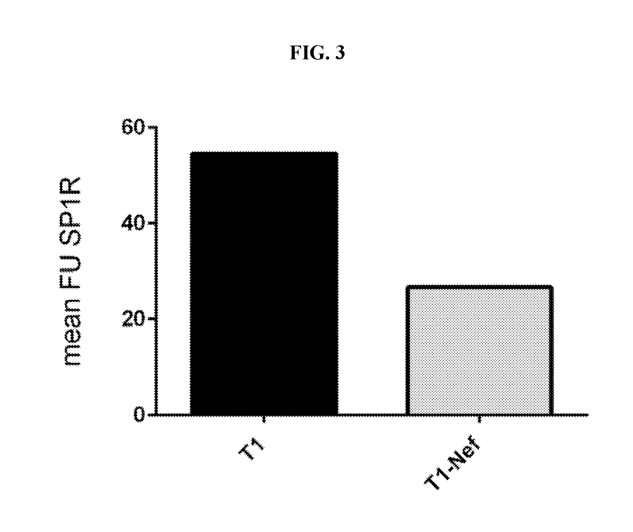

As shown in FIGS. 3 and 4, the surface receptor S1P₁ is down-regulated by HIV-Nef-induced cell signaling as determined using FACS analysis. These data demonstrate that the expression of HIV-Nef leads to downregulation and degradation of S1P₁ receptor.

### Example 4

### Beta Arrestin-TANGO Assay

The role of beta arrestin in GPCR regulation, including S1P₁, allows for the design and use of an S1P₁ Arrestin-Tango expressing cell line (TANGO construct commercially available from AddGene) to test S1P₁ signaling and its inhibitors. FIG. 6 demonstrates the design, principle and validation of the arrestin-Tango assay. FIG. 6A shows the modular design of Tango constructs (top) and the general scheme for the β-arrestin (TANGO) recruitment assay. Upon activation of the S1P₁ by an agonist, β-arrestin is recruited to the C-terminus of the receptor. This is followed by cleavage of the GPCR fusion protein at the TEV protease site. Cleavage results in the release of the tTA transcription factor, which after transport to the nucleus, activates transcription of the luciferase reporter gene.

FIG. 6B demonstrates surface expression of two selected TANGO constructs as shown by immunofluorescence using an anti-FLAG antibody. Concentration response curves of a prototypical non-orphan GPCR, the neuromedin B receptor stimulated by neuromedin B (NMB) in the TANGO assay (FIG. 6C) and in a calcium-release assay (FIG. 6D).

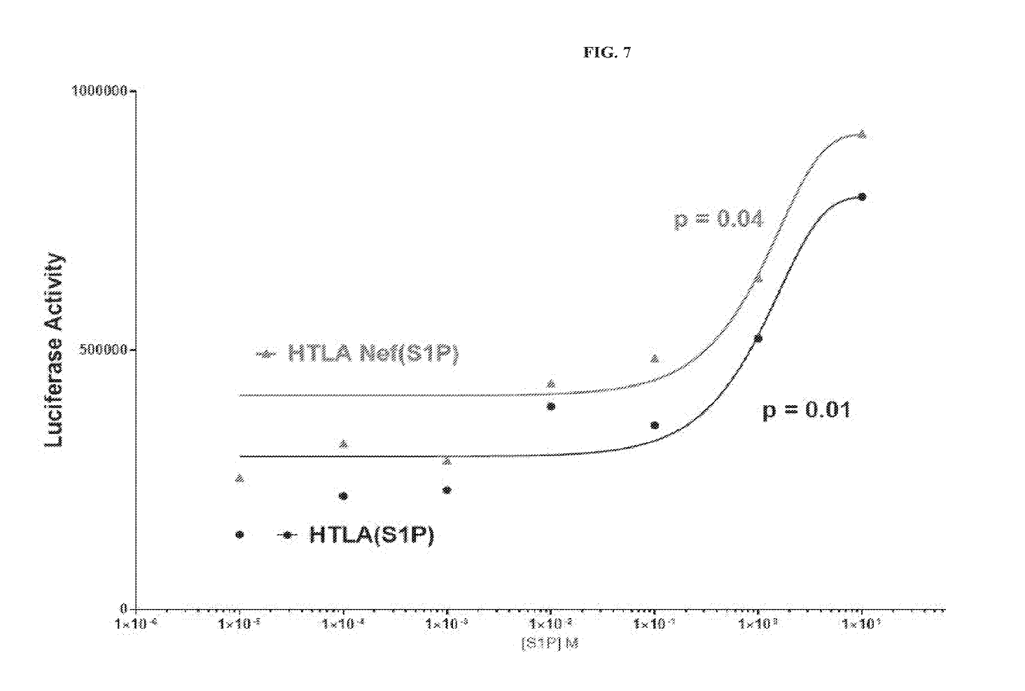

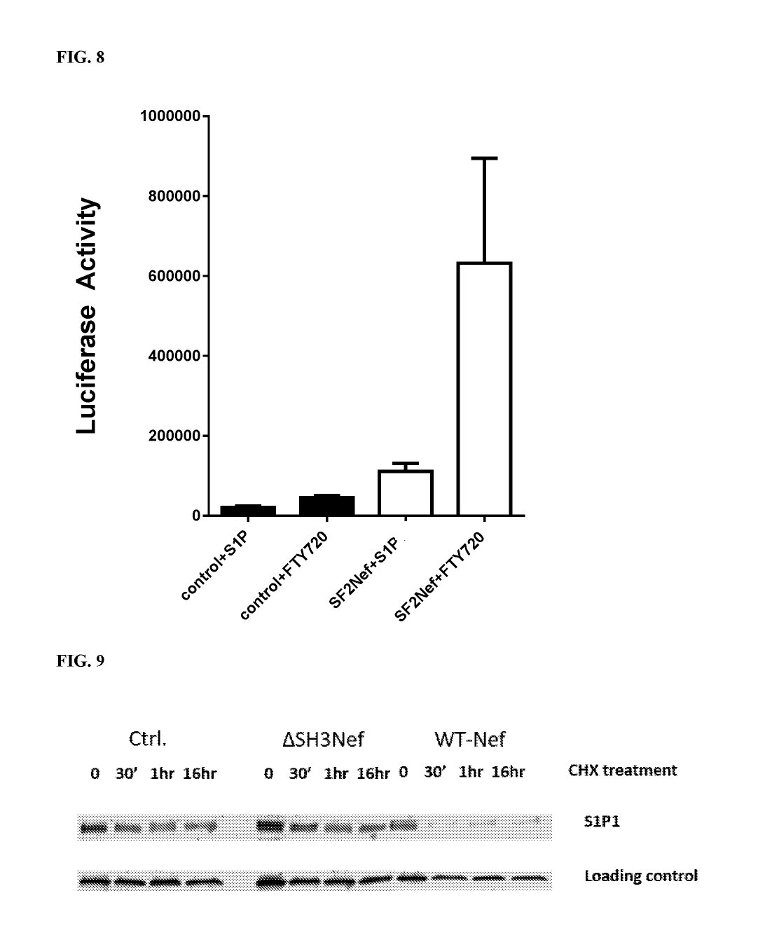

FIG. 7 demonstrates use of this assay showing Nef wt recruits beta arrestin. Specifically, HTLA cells were transfected with NEF wt and analyzed for luciferase activity in response to increasing concentrations of sphingosine-phosphate (S1P). There is more activation reflecting beta arrestin recruitment with HIV Nef transfected HTLA cells. FIG. 8 shows that HTLA cells transfected with Nef and treated with 1mM sphingosine-phosphate (S1P) and/or the synthetic agonist FTY720 (fingolimod) showed significantly more activation with HIV-Nef transfected HTLA cells either with S1P or FTY 720.

### Example 5

### Role of Src Kinase Family in S1P₁ Downregulation

This Example shows the down-regulation of S1P₁ by Nef is mediated by SH3 domain which is involved in Src kinase family signaling. Pulse chase experiments were performed. HTLA cells were transfected with Nef (wt) or Nef mutants (delta SH3) and analyzed for S1P₁ downregulation in the presence of cycloheximide (CHX) by Western blot using specific antibodies to S1P₁ and beta-actin (as loading control) in FIG. 9. HIV-Nef completely downregulated S1P₁, which is consistent with the observation of S1P₁ surface downregulation in T cells (FIG. 3). However, Nef mutant lacks the ability to downregulate S1P₁, suggesting that Src kinase signaling is involved in S1P₁ downregulation.

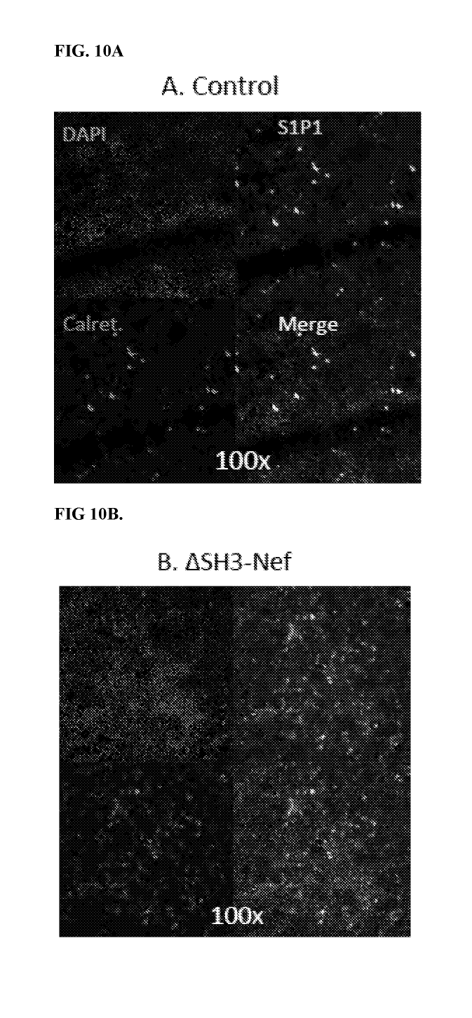

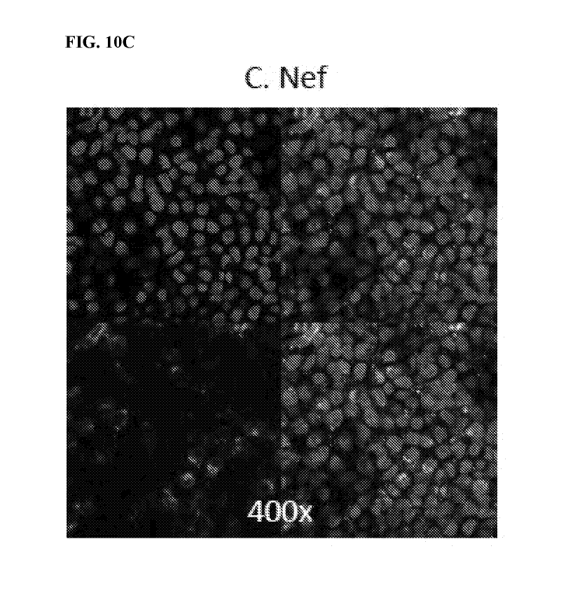

FIG. 10 demonstrates that Nef-induced S1P₁ downregulation and perinuclear recruitment depends on SH3-binding domain of Nef. HTLA cells were co-transfected with (A) control plasmids), (B) ΔSH3-Nef, (C) Nef and pS1P₁-Tango. N terminal Flag-tag of S1P₁ was stained with Flag-M2 antibodies and Laexa-488-labeled secondary antibodies. Calreticulin-RFP was used for estimating co-localization of S1P₁ with ER and Golgi. Dapi was used as nuclear staining. Therefore, antibodies or small molecules that target binding to SH3 domain of Nef could be used for treatment of HIV-infection.

We claim:

1. A method of treating a HIV infection, the method comprising: administering a therapeutically effective dose of an anti-Nef agent to a patient in need thereof; wherein the anti-Nef agent specifically targets Nef or Nef-mediated signaling, and wherein administering the therapeutic anti-Nef agent treats the HIV infection.

2. The method of claim 1, wherein the anti-Nef agent is selected from the group consisting of an anti-Nef antibody, a small molecule inhibitor of Nef or Nef-mediated signaling, and a small nucleic acid modulator of Nef or Nef-mediated signaling.

3. The method of claim 1, wherein administration of the anti-Nef agent decreases retention of HIV+ T cells in lymph nodes in the subject.

4. The method of claim 1, wherein administration of the anti-Nef agent promotes movement of HIV+ T cells into lymph and bloodstream.

5. The method of claim 2, wherein the anti-Nef agent further comprises an antibody against a cell surface protein.

6. The method of claim 2, wherein the anti-Nef agent is an agent capable of in vivo expression of an anti-Nef siRNA.

7. The method of claim 6, wherein the agent comprises a viral vector.

8. The method of claim 1, further comprising administering to the patient an antiviral agent.

9. The method of claim 8, wherein the antiviral agent is an anti-HIV agent selected from the group consisting of reverse transcriptase inhibitors, protease inhibitors viral maturation inhibitors, agents targeting the expression of HIV genes, agents targeting key host cell genes and gene products involved in HIV replication, iRNA agents, antisense RNA, vectors expressing iRNA agents or antisense RNA, PNA and antiviral antibodies.

10. A pharmaceutical composition for treating HIV infection, comprising: (1) an anti-Nef agent; and (2) a pharmaceutically acceptable carrier, wherein the anti-Nef agent reduces the level of Nef gene expression or a biological activity of the Nef protein in cells infected by HIV, resulting in migration of HIV+ T cells from lymph nodes.

11. A method for improving CD4+ T cell mediated immunity of a HIV positive patient comprising administering to the patient a therapeutically effective amount of a therapeutic anti-Nef agent, wherein said therapeutic anti-Nef agent improves CD4+ T cell mediated immunity.

12. The method of claim 11, wherein the anti-Nef agent is selected from the group consisting of an anti-Nef antibody, a small molecule inhibitor of Nef or Nef-mediated signaling, and a small nucleic acid modulator of Nef or Nef-mediated signaling.

13. The method of claim 11, wherein administration of the anti-Nef agent decreases retention of HIV+ T cells in lymph nodes in the subject.

14. The method of claim 11, wherein administration of the anti-Nef agent promotes movement of HIV+ T cells into lymph and bloodstream.

15. The method of claim 12, wherein the anti-Nef agent further comprises an antibody against a cell surface protein.

16. The method of claim 12, wherein the anti-Nef agent is an agent capable of in vivo expression of an anti-Nef siRNA.

17. The method of claim 16, wherein the agent comprises a viral vector.

18. The method of claim 11, further comprising administering to the patient an antiviral agent.

19. The method of claim 18, wherein the antiviral agent is an anti-HIV agent selected from the group consisting of reverse transcriptase inhibitors, protease inhibitors viral maturation inhibitors, agents targeting the expression of HIV genes, agents targeting key host cell genes and gene products involved in HIV replication, iRNA agents, antisense RNA, vectors expressing iRNA agents or antisense RNA, PNA and antiviral antibodies.

20. A method of treating a HIV infection, the method comprising: administering a therapeutically effective dose of an S1P₁ targeting agent able to upregulate S1P₁ to a patient in need thereof; wherein the administering the S1P₁ targeting agent treats the HIV infection.
